(19) United States
(12) Patent Application Publication
(10) Pub. No.: US 2020/0032202 A1
(43) Pub. Date: Jan. 30, 2020

(54) A MODIFIED CELL CULTURE MEDIUM AND USES THEREOF

(71) Applicant: The Board of Supervisors of Louisiana State University and Agricultural and Mechanical College, Baton Rouge, LA (US)

(72) Inventors: Nicolas Bazan, New Orleans, LA (US); Filipe Muhale, New Orleans, LA (US)

(21) Appl. No.: 16/500,972

(22) PCT Filed: Apr. 4, 2018
(86) PCT No.: PCT/US2018/026022

§ 371 Date: Oct. 4, 2019

Related U.S. Application Data
(60) Provisional application No. 62/481,419, filed on Apr. 4, 2017.

(51) Int. Cl. C12N 5/00; C12N 5/0793; A61K 35/30; A61P 25/28; A01N 1/02

(52) U.S. Cl. CPC C12N 5/0018; C12N 5/0619; A61K 35/30; A61P 25/28; C12N 2506/1307; C12N 2501/999; C12N 2506/1353; C12N 2500/36; A01N 1/0221

(57) ABSTRACT

The invention is directed to a cell culture medium composition wherein the composition comprises an effective amount of: valproic acid; a GSK-3 inhibitor; a TGFPRI inhibitor; Forskolin; a JNK inhibitor; a protein kinase C (PKC) inhibitor; a Rho-associated, coiled-coil containing protein kinase (ROCK) inhibitor; at least one omega-3 fatty acid; a nutrient source, and any combination thereof.

This application claims priority from U.S. Provisional Application No. 62/481,419, filed on Apr. 4, 2017, the entire contents of each which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### GOVERNMENT INTERESTS

This invention was made with government support under Grant No. P30 GM103340 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to a modified cell culture medium and uses thereof.

### BACKGROUND OF THE INVENTION

Forcing the expression of a defined set of transcription factors Oct4, Sox2, Klf4, Myc, (OKSM or Yamanaka factors) results in the direct reprogramming of fibroblasts into induced pluripotent stem cells (iPSC). Subsequent work showed that forced expression of three transcription factors, namely Ascl1, Brn2 (also called Pou3f2) and Mytl1, induces direct conversion of fibroblasts into neurons, work that was later confirmed by several other reports. Taken together, concomitant expression of defined sets of transcription factors is sufficient to determine the alteration of cell lineage of otherwise considered terminally differentiated cell type. This strategy suggested the potential therapeutic use of autologous cell replacement therapy.

Unfortunately, forced expression of transcription factors for iPSCs or neuron reprogramming have a strong potential to induce genomic instablility and malignancy. Therefore due to biosafety-related issues, such prior technologies have had limited clinical applications.

### SUMMARY OF THE INVENTION

Embodiments as described herein are directed towards a cell culture medium composition. An aspect of the invention is directed to a cell culture medium composition wherein the composition comprises an effective amount of: valproic acid; a GSK-3 inhibitor; a TGFβR1 inhibitor; Forskolin; a JNK inhibitor; a protein kinase C (PKC) inhibitor; a Rho-associated, coiled-coil containing protein kinase (ROCK) inhibitor; at least one omega-3 fatty acid; a nutrient source, and any combination thereof.

In embodiments, the amount of Valproic Acid ranges from about 0.5 mM to about 1 mM. For example, the amount of Valproic Acid comprises about 0.5 mM, about 0.6 mM, about 0.7 mM, about 0.8 mM, about 0.9 mM, about 1 mM.

In embodiments, the amount of a GSK-3 inhibitor ranges from about 1 μM to about 10 μM. For example, the amount of GSK-3 inhibitor comprises about 1 μM, about 2 μM, about 3 μM, about 4 μM, about 5 μM, about 6 μM, about 7 μM, about 8 μM, about 9 μM, about 10 μM. In embodiments, the GSK-3 inhibitor comprises CHIR99021, SB0216763, kenpaullone, lithium chloride, GSK-3beta inhibitor XVI, 10Z-hymenialdisine, indirubin, and the like.

In embodiments, the amount of a TGFβR1 inhibitor ranges from about 1 μM to about 10 μM. For example, the amount of a TGFβR1 inhibitor comprises about 1 μM, about 2 μM, about 3 μM, about 4 μM, about 5 μM, about 6 μM, about 7 μM, about 8 μM, about 9 μM, about 10 μM. For example, the amount of TGFβR1 inhibitor comprises about 1 μM. In embodiments, the TGFβR1 inhibitor comprises Repsox, A8-301, or an ALK-5 inhibitor, such as SB431542.

In embodiments, the amount of Forskolin ranges from about 1 μM to about 10 μM. For example, the amount of Forskolin comprises about 1 μM, about 2 μM, about 3 μM, about 4 μM, about 5 μM, about 6 μM, about 7 μM, about 8 μM, about 9 μM, about 10 μM.

In embodiments, the amount of a JNK inhibitor ranges from about 1 μM to about 10 μM. For example, the amount of JNK inhibitor comprises about 1 μM, about 2 μM, about 3 μM, about 4 μM, about 5 μM, about 6 μM, about 7 μM, about 8 μM, about 9 μM, about 10 μM. In embodiments, the JNK inhibitor comprises SP600125.

In embodiments, the amount of a PKC inhibitor ranges from about 1 μM to about 10 μM. For example, the amount of PKC inhibitor comprises about 1 μM, about 2 μM, about 3 μM, about 4 μM, about 5 μM, about 6 μM, about 7 μM, about 8 μM, about 9 μM, about 10 μM. In embodiments, the PKC inhibitor comprises GO6983 or H7.

In embodiments, the amount of a ROCK inhibitor ranges from about 1 μM to about 10 μM. For example, the amount of ROCK inhibitor comprises about 1 μM, about 2 μM, about 3 μM, about 4 μM, about 5 μM, about 6 μM, about 7 μM, about 8 μM, about 9 μM, about 10 μM. In embodiments, the ROCK inhibitor comprises Y-27632, 1-(5-Isoquinolinesulfonyl) homopiperazine, N-Benzyl-2-(pyrimidin-4-ylamino)thiazole-4-carboxamide, (+)-(R)-trans-4-(1-aminoethyl)-N-(4-pyridyl) cyclo-hexanecarboxamide dihydrochloride) and N-{(3R,4R)-4-[4-(2-Fluoro-6-hydroxy-3-methoxy-benzoyl)-benzoylamino ]-azep-an-3-yl}1-4-hydroxy-3,5-dimethyl-benzamide.

In embodiments, the amount of an omega-3 fatty acid ranges from about 1 μM to about 50 μM. For example, the amount of an omega-3 fatty acid comprises about 1 μM, about 5 μM, about 10 μM, about 20 μM, about 25 μM, about 30 μM, about 35 μM, about 40 μM, about 45 μM, or about 50 μM.

In embodiments, the omega-3 fatty acid is selected from at least one of Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA), Clupanodonic acid, Docosahexaenoic acid (DHA), Tetracosapentaenoic acid, Tetracosahexaenoic acid (Nisinic acid), or precursors thereof. Embodiments as described herein comprise culture medium compositions comprising any combination of omega-3 fatty acids.

In embodiments, the effective amount of EPA ranges from 1 μM to about 50 μM. For example, the effective amount of EPA is about 10 μM.

In embodiments, the effective amount of DHA ranges from about 1 μM to about 50 μM. For example, the effective amount of DHA is about 20 μM.

In embodiments, the nutrient source is selected from at least one of DMEM, IDMEM, MEM, M199, RPMI 1640, Ham's F12, DMEM/F-12, Ham's F10, McCoy's 5A, NCTC 109, and NCTC 135.

An aspect of the invention is directed towards a method of promoting neuronal cell conversion. In embodiments, the method comprises obtaining a plurality of non-neuronal cells; admixing the plurality of non-neuronal cells with the cell culture medium composition as described in embodiments herein; and culturing the admixture for a period of time sufficient for the non-neuronal cells to transdifferentiate into neuronal or neuronal-like cells. Neuronal-like cells, for example, can be characterized by cell bodies with a network of long thin processes, and can present immunoreactivity towards antibodies specifically directed against neuronal type cells, or a combination thereof.

An aspect of the inventions is directed towards a method of inducing neuronal cell differentiation. In embodiments, the method comprises obtaining a plurality of non-neuronal cells; admixing the plurality of non-neuronal cells with the cell culture medium composition as described in embodiments herein; and culturing the admixture for a period of time sufficient to induce differentiation of non-neuronal cells into neuronal cells.

An aspect of the invention is directed to a method of treating a subject afflicted with a disease characterized by neuronal death, neuronal injury, or both. In embodiments, the method comprises obtaining a plurality of non-neuronal cells; admixing the plurality of non-neuronal cells with the cell culture medium composition of embodiments as described herein; culturing the admixture for a period of time sufficient to induce differentiation of non-neuronal cells into neuronal cells; and administering the differentiated neuronal cells to the subject in need thereof.

Embodiments as described herein can further comprise administering the neuronal cells to a subject afflicted with a disease characterized by neuronal death, neuronal injury, neuroinflammation, or any combination thereof. For example, the disease comprises a neurological disorder, non-limiting examples of which comprise stroke, Alzheimer's Disease, Parkinson's Disease, Amyotrophic Lateral Sclerosis (ALS), Multiple Sclerosis (MS), Frontotemporal dementia, and Huntington's disease. In embodiments, the neurological disorder comprises a neurodegenerative disorder.

In embodiments, the differentiation or transdifferentiation of non-neuronal cells into neuronal cells is detected by immunophenotyping. For example, the immunophenotyping comprises detecting or measure neuronal markers, non-limiting examples of which comprise NeuN, β-III-tubulin, (TujI), Map2, Synapsin I, Synaptophysin, and PSD-95.

In embodiments, the plurality of non-neuronal cells have been isolated from the subject. For example, the plurality of non-neuronal cells have been isolated from the subject; the non-neuronal cells are transdifferentiated into neuronal cells using embodiments as described herein, and the neuronal cells are administered to the subject.

In embodiments, the transdifferentiated neuronal cells comprise viable neurons and/or function as functional neurons. Non-limiting examples of neuronal functions comprise ability to establish synapses, for example, as revealed by immunoreactivity to markers such as PSD-95 and synaptophysin. In embodiments, functional neurons are identified, confirmed and/or detected using immunophenotyping, for example using neuronal markers.

In embodiments, the admixture is cultured for about 1 day, about 2 days, about 3 days, about 4 days, about 5 days, about 6 days, about 7 days, about 8 days, about 9 days, or about 10 days. In embodiments, the admixture is cultured for less than 21 days. In embodiments, the cell culture medium composition can be removed and replaced with fresh cell culture medium composition one more times during culturing.

In embodiments, the non-neuronal cells comprise fibroblasts or mesenchymal stems cells. For example, the non-neuronal cells can be derived from bone marrow or adipose tissue.

An aspect of the invention is directed towards an in vitro culture comprising an isolated population of in vitro transdifferentiated cells and a cell culture medium, for example the medium as described herein, supplemented with DHA and/or EPA, wherein greater than about 80%, about 85% about, 90%, about 91%, about 92%, about 93%, about 94%, about 95%, about 96%, about 97%, about 98%, about 99% of the cells in the population are neuronal cells. In embodiments, the cell culture medium is serum free.

Further, an aspect of the invention is directed towards a cytoprotective conditioned media composition characterized by being the product of culturing transdifferentiated neurons in the media as described herein for a period of time. In embodiments, the period of time comprises about 1 day, about 2 days, about 3 days, about 4 days, about 5 days, about 6 days, about 7 days, about 8 days, about 9 days, about 10 days, or greater than 10 days.

In embodiments, the cytoprotective conditioned media comprises a serum-free cell culture media. In other embodiments, the cytoprotective conditioned media is cell-free or substantially cell free.

The cytoprotective conditioned media can further comprise neuroprotective or cytoprotective compositions, such as docosanoids, NPD1, maresins, resolvins, or a combination thereof

Aspects of the invention are also directed towards a method of treating a subject afflicted with a disease characterized by neuronal death, neuronal injury, or both comprising administering a therapeutically effective amount of the cytoprotective conditioned media.

Aspects of the invention are also directed towards a method of delay or preventing neuronal degeneration in a subject suffering from a neurological disorder comprising administering to the subject a therapeutically effective amount of the cytoprotective conditioned media.

Aspects of the invention are directed towards a method of treating a subject afflicted with a neurological disorder comprising administering a therapeutically effective amount of the cytoprotective conditioned media.

Non-limiting examples of neurological disoders that can be treated by the cytoprotective conditioned media include Alzheimer's Disease, Parkinson's Disease, Amyotrophic Lateral Sclerosis (ALS), Multiple Sclerosis (MS), Frontotemporal dementia, and Huntington's disease. For example, the cytoprotective conditioned media can reduce or ameliorate the symptoms associated with the neurological disorders.

Further, aspects of the invention are directed towards a method of making a cytoprotective conditioned media comprising culturing transdifferentiated neurons in a media as described herein a perior of time sufficient for the media to develop cytoprotective properties. In embodiments, the conditioned media is a neuroprotective conditioned media.

In embodiments, the cytoprotective properties of the media is indicated by the presence of at least cytoprotective agent, such as a docosanoid, a maresin, a resolvin, or NPD1.

Other objects and advantages of this invention will become readily apparent from the ensuing description.

### BRIEF DESCRIPTION OF THE FIGURES

FIG. 1 shows the baseline levels of neuronal cultures assess by the alamarBlue assay. Fluorescence intensities for the alamarBlue assay measured with the molecular devices M5 plate reader (excitation 540 nm/emission 590 nm) were converted into % cells by dividing the actual fluorescence against the mean fluorescence of the untreated control cells×100. Student's t-test was used to determine statistical significance p value set at p≤0.05. (A) Cortical neurons, (B) Hippocampal neurons. Normally growing rat cortical neurons presented similar levels of cell density irrespective to the feeding media. In the hippocampal neurons, although statistically significant, cell density without insult in between treatments remained similar. However, relative to untreated control, three days after injury by OGD, in both cortical and hippocampal rat neuronal cultures, control media and IM-conditioned media groups had a drastic and statistically significant reduction in the percentage of viable cells. Cell death following OGD was strongly rescued by the treatment with conditioned BM-media.

FIG. 2 shows three days after OGD: density of neuronal cultures assessed by the alamarBlue assay. (A) Cortical neurons, (B) Hippocampal neurons. Statistical analysis (*):p≤0.05 vs OGD control. (C) Bright-field images of rat cortical neurons.

FIG. 3 shows immunofluorescence analysis with neuronal markers. Stem cells used to rescue injured rat neurons were trans-differentiated from human skin fibroblasts into neurons with (A) induction media or IM, and (B) induction media plus DHA and EPA, or BM; or (c) are representative images of rat primary neurons.

FIG. 4 show the use of neuronal-type chemical induction to both types of mesenchymal stem cells derived from rat bone marrow or from the human adipose tissue display. By five days post-induction, clear morphological features of neuronal-type transdifferentiation become evident in both types of stem cells.

FIG. 5 shows a schematic of metabolism of DHA

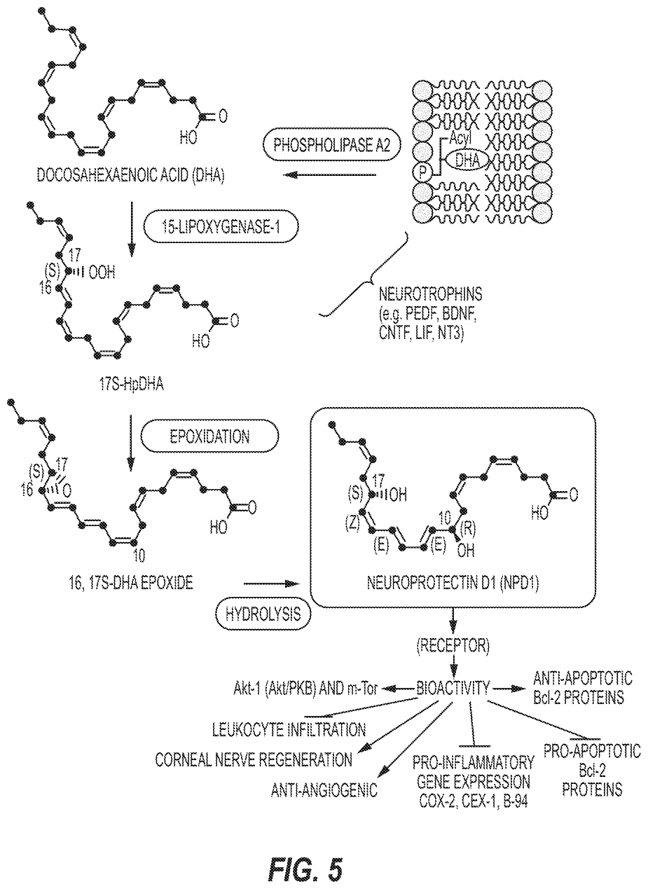

FIG. 6 shows a schematic of biosynthesis of neuroprotection Dl.

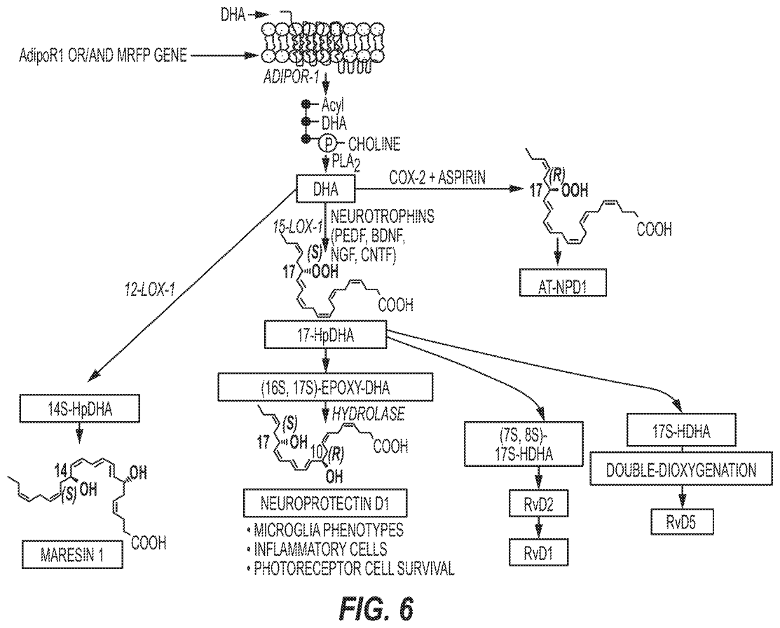

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is used consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” can be that the process includes at least steps a, b and c. Wherever the terms “a” or “an” are used, “one or more” is understood, unless such interpretation is nonsensical in context.

The term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Omega-3 Fatty Acids

Omega-3 fatty acids are polyunsaturated fatty acids that have the final double bond in the hydrocarbon chain between the third and fourth carbon atoms from the methyl end of the molecule. Embodiments as described herein, for example the cell culture media composition, comprise one or more omega-3 fatty acids as a component thereof. Non-limiting examples of omega-3 fatty acids include, 5,8,11,14,17-eicosapentaenoic acid (EPA), 4,7,10,13,16,19-docosahexanoic acid (DHA), 7,10,13,16,19-docosapentanoic acid (DPA), and α-linolenic acid (ALA), or a combination thereof. Referring to Example 1 and Example 2, for example, an embodiment comprises a modified cell culture medium comprising EPA and DHA.

Omega-3 fatty acids are important for normal metabolism. Mammals are unable to synthesize omega-3 fatty acids, but can obtain the shorter-chain omega-3 fatty acid ALA (18 carbons and 3 double bonds) through diet and use it to form the more important long-chain omega-3 fatty acids, EPA (20 carbons and 5 double bonds) and then from EPA, the most crucial, DHA (22 carbons and 6 double bonds). The ability to make the longer-chain omega-3 fatty acids from ALA may be impaired in aging.

In embodiments as described herein, the omega-3 fatty acid is selected from at least one of Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA), Clupanodonic acid, Docosahexaenoic acid (DHA), Tetracosapentaenoic acid, Tetracosahexaenoic acid (Nisinic acid), or precursors thereof. Embodiments as described herein comprise any combination of omega-3 fatty acids.

### Cell Culture Media

Embodiments as described herein are directed towards a cell culture medium composition, the composition comprising an effective amount of: valproic acid; a GSK-3 inhibitor; a TGFβR1 inhibitor; Forskolin; a JNK inhibitor; a protein kinase C (PKC) inhibitor; a Rho-associated, coiled-coil containing protein kinase (ROCK) inhibitor; at least one omega-3 fatty acid; and a nutrient source. Embodiments are further directed towards any combination thereof.

The term “culture”, “cell culture” can refer to a cell population that is suspended in a medium under conditions suitable to survival and/or growth of the cell population. In embodiments, these terms as used herein also refer to the combination comprising the cell population and the medium in which the population is suspended. In certain embodiments, the cell culture is a mammalian cell culture. For example, the cell culture is a human cell culture.

“Culturing,” can refer to maintaining cells under conditions in which they can proliferate, differentiate, and avoid senescence. For example, in the present invention, cultured non-neuronal cells, such as mesenchymal stem cells, proliferate and differentiate into cells of an neuronal cell lineage. Referring to Example 1, for example, culturing non-neuronal cells in cell culture media supplemented with omega-3 fatty acids such as DHA and EPA dramatically increased transdifferentiation efficiency of the non-neuronal cells into cells of the neuronal lineage.

The terms “medium”, “cell culture medium”, “culture medium” canrefer to a solution containing nutrients that nourish growing cells. In certain embodiments, the culture medium is useful for growing mammalian cells. Typically, a culture medium provides essential and non-essential amino acids, vitamins, energy sources, lipids, and trace elements required by the cell for minimal growth and/or survival. A culture medium may also contain supplementary components (see discussion of “Supplementary components” below) that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In certain embodiments, a medium is advantageously formulated to a pH and salt concentration optimal for cell survival and proliferation.

The term “supplementary components” can refer to components that enhance growth and/or survival above the minimal rate, non-limiting examples of which comprise hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In certain embodiments, supplementary components are added to the initial cell culture. In certain embodiments, supplementary components are added after the beginning of the cell culture.

The term “defined medium” can refer to a medium in which the composition of the medium is both known and controlled.

The term “nutrient source” can refers to a composition, including the source itself, that nourishes growing mammalian cells. Non-limiting examples of nutrient sources comprise DMEM, IDMEM, MEM, M199, RPMI 1640, Ham's F12, DMEM/F12, Ham's F10, McCoy's 5A, NCTC 109, and NCTC 135.

The term “effective amount” can refer to the amount of a compound (e.g., DHA, EPA) sufficient to effect beneficial or desired results, such as being neuroprotective (see Example 2, for example) or transdifferentiation (see Example 1 and Example 3, for example). For example, “effective amount” can refer to the amount of DHA and EPA in a culture media sufficient to promote neuronal cell conversion or induce neuronal cell differentiation. In embodiments, the desired or beneficial result is confirmed by immunophenotyping as described herein. The effective amount of a compound sufficient to effect a beneficial or desired result, such as transdifferentiation of a non-neuronal cell into a neuronal lineage, can be dependent on the non-neuronal cell to be cultured in the cell culture medium. Referring to Example 1, for example, supplementation of cell culture medium with 20 μM EPA and 40 μM DHA was sufficient to transdifferentiate fibroblasts into cells of the neuronal lineage. Referring to Example 3, supplementation of cell culture medium comprising 10 μM EPA and 20 μM DHA was sufficient to transdifferentiate bone marrow stem cells and human adipose tissue stem cells into cells of the neuronal lineage. Still further, Example 2 describes a modified cell culture medium comprising an amount of two essential fatty acids, specifically 10 μM EPA and 20 μM DHA, that is neuroprotective to stroke-like conditions.

An “inhibitor” can refer to any compound, biologic, or substance which directly or indirectly inhibits the activity of a protein. For example, the inhibitor can be a GSK-3 inhibitor, which inhibits the acitivity of GSK-3. Similarly, the inhibitor can be a TGFβR1 inhibitor, an ALK-5 inhibitor, a JNK inhibitor, a PKC inhibitor, or a ROCK inhibitor.

### Methods of Transdifferentiation

Embodiments as described herein are directed towards a method of promoting neuronal cell conversion of non-neuronal cells. For example, the non-neuronal cells are cultured in a cell culture medium composition as described in embodiments herein for a period of time sufficient for the non-neuronal cells to transdifferentiate into neuronal or neuronal-like cells. Referring to Example 1, for example, supplementation of cell culture medium with 20 μM EPA and 40 μM DHA was sufficient to transdifferentiate fibroblasts into cells of the neuronal lineage.

Similarly, embodiments as described herein are directed towards a method of inducing neuronal cell differentiation of non-neuronal cells. Referring to Example 3, for example, supplementation of cell culture medium with 10 μM EPA and 20 μM DHA was sufficient to induce differentiation of mesenchymal stem cells derived from human adipiase tissue or mouse bone marrow in cells of the neuronal lineage.

As used herein, “transdifferentiation” or “conversion” can refer to the process by which cell types of one lineage, including precursor or progenitor cells (i.e., stem cells) pre-committed to cell types of one lineage, differentiate into specific cell types of another lineage, e.g., mesenchymal stem cells transdifferentiate into neuronal cells. Transdifferentiated cells can be identified by their patterns of gene expression and cell surface protein expression, such as by immunophenotyping. Typically, cells of an neuronal lineage express genes such as, for example, NeuN, β-III-tubulin, (TujI), Map2, Synapsin I, Synaptophysin, and PSD-95.

“Differentiation” can refer to the process whereby relatively unspecialized cells (e.g., embryonic cells, stem cells) acquire specialized structural and/or functional features characteristic of mature cells. Similarly, “differentiate” can refer to this process. During differentiation, cellular structure alters and tissue-specific proteins appear. The term “differentiated neuronal cell” can refer to cells expressing a protein characteristic of a specific neuronal cell type. For example, differentiated neuronal cell types can comprise those that express immunoreactivity to two or more neuronal lineage markers, non-limiting examples of which comprise NeuN, β-III-tubulin, (Tujl), Map2, Synapsin I, Synaptophysin, and PSD-95.

“Cell viability”can refer to the ability of cells in culture to survive under a given set of culture conditions or experimental variations. The term can also refer to that portion of cells which are alive at a particular time in relation to the total number of cells, living and dead, in the culture at that time.

“Neuron” can refer to a nerve cell, also termed a neuronal cell.

“Neuronal cell” can refer to a cell that is a morphologic and functional unit of the nervous system. The cell can comprise a nerve cell body, the dendrites, and the axon. The terms neuron, nerve cell, neuronal, neurone, and neurocyte can be used interchangeably. Non-limiting examples of neuronal cell types comprise a typical nerve cell body showing internal structure, a horizontal cell (of Cajal) from cerebral cortex; Martinottic cell, biopolar cell, unipolar cell, Pukinje cell, and a pyramidal cell of motor area of cerebral cortex.

“Neural” can refer to anything associated with the nervous system, such as cells of the nervous system. For example, the term “neural cells” can include, but is not limited to, neurons and glia, including astrocytes. “Isolated neural cells” can refer to neural cells isolated from brain.

As used herein, the term “non-neuronal cell” can refer to any viable cell not of the neuronal lineage. Non-neuronal cells suitable for use in the methods of the present invention comprise those that are capable of transdifferentiating into cells of a neuronal lineage. Non-limiting examples of suitable non-neuronal cells for use in the methods of the present invention comprise fibroblasts,; stem cells, such as bone marrow- and adipose tissue-derived mesenchymal stem cells; muscle cells (smooth, striated, or cardiac); cartiledge cells; skin cells; bone cells; endothelial cells; epithelial cells (which can be from different tissues); fat cells (adipocytes); cells of a gland; or blood cells (such as red blood cells (erythrocytes) or white blood cells (leukocytes); platelets.

A “stem cell,” as used herein, can be any self-renewing pluripotent cell or multipotent cell or progenitor cell or precursor cell that is capable of differentiating into multiple cell types. Stem cells suitable for use in the methods of the present invention include those that are capable of transdifferentiating into cells of a neuronal lineage. Non-limiting examples of suitable stem cells for use in the methods of the present invention comprise somatic stem cells, such as neural stem cells, hematopoietic stem cells, or mesenchymal stem cells (such as those isolated from bone marrow and adipose tissue) and/or embryonic stem cells.

Mesenchymal stem cells (MSC), for example, are capable of differentiating into the mesenchymal cell lineages, such as bone, cartilage, adipose, muscle, stroma, including hematopoietic supportive stroma, and tendon, and play important roles in repair and regeneration (see, e.g., Olsen, 2000, supra). MSCs are identified by specific cell surface markers which are identified with unique monoclonal antibodies as described in e.g., U.S. Pat. No. 5,643,736, which is incorporated by reference in its entirety.

“Immunophenotyping” can refer to the detection of antigenic determinants (which are unique to particular cell types) on the surface of cells, such as transdifferentiated neuronal cells, using antigen-specific antibodies, such as monoclonal antibodies or polyclonal antibodies, that have been labeled, such as with a fluorescent dye or fluorochrome (e.g., phycoerythrin [PE] or fluorescein isothiocyanate [FITC]). The labeled cells can then be analyzed, such as by using a flow cytometer which categorizes individual cells according to size, granularity, fluorochrome, and intensity of fluorescence.

In embodiments, neuronal and stem cells cultures can be characterized by immunofluorescence staining. For example, primary antibodies such as the class-III-β tubulin monoclonal antibody, the rabbit anti-glial fibrillary acidic protein, the doublecortin, anti-NeuN antibodies, anti-Map2 antibodies, anti-synapsin antibodies, anti-synaptophysin antibodies, can first bind their cellular antigens. Primary antibodies bound to their cellular antigens can be detected with secondary antibodies, such as AF488 conjugated donkey anti-mouse IgG, AF647 conjugated donkey anti-rabbit, and nounterstained with Hoechst 33258.

### Cytoprotective Conditioned Media

Embodiments as described herein comprise a cytoprotective “conditioned” media that comprises secretions from the transdifferentiated neurons. In embodiments, this conditioned media is cytoprotective. For example, the conditioned media is neuroprotective (i.e., protects against cell death from in a model of stroke).

As described herein, non-neuronal cells are transdifferentiated into cells of a neuronal lineage using a media described herein, such as a cell culture media comprising DHA, EPA, or a combination thereof. Cells of a neuronal lineage transdifferentiated with media described herein, such as the cell culture media comprising DHA, EPA, or a combination thereof, excrete products into the conditioned media that are cytoprotective. This effect is not seen in cells transdifferentiated with basic media without an omega-3 fatty acid, such as DHA and/or EPA (referred to herein as IM). DHA is the precursor of docosanoids, which are neuroprotective, pro-homeostatic mediators. Non-limiting examples of such mediators comprise NPD1, maresins and resolvins.

The term “cytoprotective” refers to the ability of compositions or agents, for example the Conditioned Media or agents therein, natural or not, to maintain the interactions of cells with each other or with the other tissues, to protect cells from the degeneration phenomena leading to a loss of cell function or to undesirable cell activities, with or without cell death, and/or from cell dysfunctions and/or from the degenerative diseases or disorders leading to these cell dysfunctions, said dysfunctions or said diseases or conditions leading or not leading to cell death.

For example, the term “neuroprotective” refers to the same properties of said compositions or agents but specifically for cells of the nervous system (“neuroprotective”).

This cytoprotective conditioned media, for example, can act in an autocrine and/or paracrine fashion on injured cells, such as injured neurons. For example, without wishing to be bound by theory, the paracrine action of the conditioned media can be one of the modalities for the media's neuroprotective bioactivity on a rat model of neuronal injury.

### Therapeutic Methods

Embodiments as described herein comprise methods of treating a subject afflicted with a disease characterized by neuronal death, neuronal injury, neuroninflammation, or any combination thereof. For example, the subject is administered transdifferentiated neuronal cells as described herein.

In embodiments, the non-neuronal cells are isolated from the subject in need thereof or obtained from a donor subject, the non-neuronal cells are transdifferentiated into cells of a neuronal lineage, for example by using the cell culture medium as described herein, and the neuronal cells are administered or transplanted back into the subject in need thereof. In such embodiments, the risk of rejection, such as immune rejection, may be limited.

As used herein, the terms “subject” and “patient” are used interchangeably and can refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject or patient is a human.

For example, a “patient in need thereof” can refer to a patient suffering from a neurological disorder, such as a neuronal injury or neurodegeneration, that occurs as a result of an acute event (e.g., a stroke or central nervous system injury/trauma) or as a result of a chronic neurodegenerative process. Said acute neuronal injury or progressive neurodegenerative process may affect the neurons of the central nervous system (CNS), composed of the brain and spinal cord, or the peripheral nervous system, and may eventually progress to neuronal death.

“Treating” and “treatment” as used herein can refer to administering to a subject a therapeutically effective amount of a composition, such as a population of transdifferentiated neuronal cells, so that the subject has an improvement in the disease or condition. The improvement is any observable or measurable improvement. Thus, one of skill in the art understands that a treatment may improve the patient's condition, but may not be a complete cure of the disease. Treating may also comprise treating subjects at risk of developing a disease and/or condition.

“Administer” can refer to causing a composition of the present invention, such as a population of transdifferentiated neuronal cells, to be delivered to a subject in such a manner that the composition can be therapeutically effective for its intended purpose. For example, “administer” includes, without limitation, the application of the compositions into the blood or brain tissue of a subjection, i.e., by injection.

Embodiments as described herein may be used to delay or prevent the onset of disease or symptoms of disease, or to delay or prevent the progression of disease. The term “preventing” as used herein can refer to minimizing, reducing or suppressing the risk of developing a disease state or parameters relating to the disease state or progression or other abnormal or deleterious conditions. For example, a population of transdifferentiated neuronal cells may delay or prevent the progression of a neurodegenerative disease, such as Alzheimer's disease.

According to the invention, “symptoms” can refer to one or more biological and/or physiological sequelae, including but not limited to memory loss, personality changes, problems with movement, weakness, or poor balance or coordination. Some common symptoms of degenerative disorders of the brain are memory loss, personality changes, problems with movement, weakness, or poor balance or coordination.

“Neuronal injury” can refer to the damage to the function or structure (e.g., cytoskeletal damage) of neurons as a result of an insult (e.g., exposure to neurotoxins) or trauma (e.g., traumatic brain injury, concussive head trauma) to the nervous system. Neuronal injury is associated with, for instance: stroke, ischemic events (e.g., brain ischemia, ischemia of the eyes), seizures of diverse etiology (epileptic, associated with brain injury, of genetic origin), spinal cord injury or trauma, brain damage due to drugs of abuse, or excitotoxic insults of diverse nature.

In embodiments, the “neuronal injury” or “neuronal cell death” can be the result of a stroke. For example, “stroke” can refer to any acute, clinical event related to the impairment of cerebral circulation. The terms “acute cerebral ischemia” and “stroke” can be used interchangeably.

As used herein, the term “neurodegeneration” can refer to the progressive loss of individual or collective structure or function of neurons, up to and including the death of neurons that is associated with many neurodegenerative diseases.

For example, “neurodegenerative disease(s)” or “neurodegenerative disorder” can refer to medical conditions that are characterized clinically by their insidious onset and chronic progression. In many instances, particular parts of the brain, spinal cord, or peripheral nerves functionally fail and the neurons of the dysfunctional region die. Neuroanatomically localizable functional impairment and “neurodegeneration” associate with recognizable syndromes or conditions that are ideally distinct, although in clinical and even neuropathologic practice substantial overlap exists. Neurodegenerative diseases are often categorized by whether they initially affect cognition, movement, strength, coordination, sensation, or autonomic control.

Frequently, however, patients will present with symptoms and signs referable to more than one system. Either involvement of several systems can occur concomitantly, or else by the time the patient has functionally declined enough to seek medical attention multiple systems have become involved. In many cases, the diagnosis of a neurodegenerative disease cannot be critically ‘confirmed’ by a simple test.

The term “neurodegenerative” can refer to the loss of neurons that cause disease.

However, without being bound by theory, neuronal demise can be the final stage of a preceding period of neuron dysfunction. It is difficult to know whether clinical decline associates with actual neuron loss, or with a period of neuron dysfunction that precedes neuron loss. Also, particular neurodegenerative diseases are etiologically heterogeneous. In addition to syndromically defining neurodegenerative diseases by what neuro-anatomical system is involved, these disorders are broken down along other clinical lines. Early (childhood, young adulthood, or middle aged adulthood) versus late (old age) onset is an important distinction. Some clinically similar neurodegenerative diseases are sub-categorized by their age of onset, despite the fact that at the molecular level different forms of a particular disease may have very little in common. Sporadic onset versus Mendelian (genetic) inheritance constitutes another important distinction, and many named neurodegenerative diseases have both sporadic (wherein Mendelian inheritance is not recognizable) and Mendelian subtypes.

Non-limiting examples of neurodegenerative diseases comprise dementia, for example Alzheimer's Disease, multi-infarct dementia, AIDS-related dementia, and Fronto temperal Dementia; neurodegeneration associated with cerebral trauma; Parkinson's Disease; Amyotropic Lateral Sclerosis (ALS); Multiple Sclerosis (MS); Huntington's disease; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; and multi-system atrophy.

Neurodegenerative diseases may present with memory loss or personality change, non-limting examples of which comprise Alzheimer's disease, Frontotemporal Dementias, Dementia with Lewy Bodies, Prion diseases.

Neurodegenerative diseases may present as problems with movement, non-limiting examples of which comprise Parkinson's disease, Huntington's disease, Progressive Supranuclear Palsy, Corticobasal Degeneration, Multiple System Atrophy.

Neurodegenerative diseases may present as weakness, non-limitng examples of which comprise amyotrophic lateral sclerosis, inclusion body myositis, degenerative myopathies.

Neurodegenerative diseases can present as poor balance, non-limting examples of which comprise the spinocerebellar atrophies.

Disorders of myelin include multiple sclerosis and Charcot-Marie-Tooth disease.

The term “motor neuron diseases” (MNDs) refers to a group of progressive neurological disorders that destroy motor neurons, the cells that control essential voluntary muscle activity such as speaking, walking, breathing, and swallowing. The best-known motor neuron disease is amyotrophic lateral sclerosis (ALS). Normally, messages from nerve cells in the brain (called upper motor neurons) are transmitted to nerve cells in the brain stem and spinal cord (called lower motor neurons) and from them to particular muscles. Upper motor neurons direct the lower motor neurons to produce movements such as walking or chewing. Lower motor neurons control movement in the arms, legs, chest, face, throat, and tongue. Spinal motor neurons are also called anterior horn cells. Upper motor neurons are also called corticospinal neurons.

When there are disruptions in the signals between the lowest motor neurons and the muscle, the muscles do not work properly; the muscles gradually weaken and may begin wasting away and develop uncontrollable twitching (called fasciculations). When there are disruptions in the signals between the upper motor neurons and the lower motor neurons, the limb muscles develop stiffness (called spasticity), movements become slow and effortful, and tendon reflexes such as knee and ankle jerks become overactive. Over time, the ability to control voluntary movement can be lost. The following is a list of the most common MNDs: Amyotrophic lateral sclerosis (ALS), also called Lou Gehrig's disease, progressive bulbar palsy, also called progressive bulbar atrophy, pseudobulbar palsy, Primary lateral sclerosis (PLS), progressive muscular atrophy, spinal muscular atrophy (SMA) and some of its variants (e.g., SMA type I, also called Werdnig-Hoffmann disease, SMA type II, SMA type III also called Kugelberg-Welander disease, congenital SMA with arthrogryposis, Kennedy's disease, also known as progressive spinobulbar muscular atrophy and post-polio syndrome (PPS)).

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

A chemically-based cell culture for direct neural conversion from other cells

We have developed an induction media (IM) (1) to which we added docosahexaenoic acid (DHA) and eicosapentaenoic acid (EPA), and within a seven-week period we obtained remarkable transdifferentiation from non-neural cells into neurons.

(1) Hu W, et al. Direct Conversion of Normal arid Alzheimer's Disease Human Fibroblasts into Neuronal Cells by Small Molecules. Cell Stem Cell. 2015; 17: 204-212.

(2) Katakura M, et al. Omega-3 polyunsaturated Fatty Acids enhance neuronal differentiation in cultured rat neural stem cells. Stem Cells Int. 2013; 490476.

Embodiments as described herein comprise a new chemically-based cell culture medium for direct neuronal conversions of non-neuronal cells, such as fibroblasts (rodents or humans, for example), mesenchymal and adipose tissue-derived stem cells that hold promise for the treatment of disorders, such as neurodegenerative disorders.

Takahashi and Yamanaka (1) demonstrate that forcing the expression of a defined set of transcription factors (Oct4, Sox2, Klf4, Myc, also known as OKSM or Yamanaka factors) results in the direct reprogramming of fibroblasts into induced pluripotent stem cells (iPSC). Subsequent work showed that forced expression of three transcription factors, namely Ascl1, Brn2 (also called Pou3f2) and Mytl1, induces direct conversion of fibroblasts into neurons (2); this work of confirmed by several other reports (3, 4, 5, 6). Therefore concomitant expression of defined sets of transcription factors is enough to determine the alteration of cell lineage of otherwise considered terminally differentiated cell type. This strategy opened the potential for the use of autologous cell replacement therapy, particularly in neurodegenerative diseases.

Unfortunately, forced expression of transcription factors iPSCs or neuron reprogramming have a strong potential to induce genomic instablility and malignancy (7-13). Therefore due to biosafety-related issues this technology has limited clinical application. In an effort to attempt to mitigate these biocompatibility/health-related concerns in cell reprogramming, Hou P, et al., 2013 (14) developed a chemical cocktail for fibroblasts transdifferentiation into iPSCs. Using a similar approach Hu et al., 2015 (15) created a chemical cocktail made of 7 molecules (induction media or IM) is hindered by low efficiency rates of cell fate conversion about 20%. Conceptually, chemically induced neuronal cells seem to be a better alternative for clinical application. Therefore there is a need to identify the molecular hurdles that limit transdifferentiation efficacy and to alter their activity pharmacologically.

First, docosahexaenoic acid (DHA), a 22:6 n-3 fatty acid, and its related fatty acid eicosapentaenoic acid, (EPA, 20:5 n-3) influences restoration of neuronal genesis, maturation, and functionality after insult (16-18) by producing the on-demand stress-response prohealing molecule neuroprotection Dl (19-21). Secondly, Katakura M, et al., 2013 (22) showed DHA/EPA maximize neuronal differentiation in cultured rat neural stem cells. Last by using candidate gene approach, we hypothesized, tested and found that DHA/EPA are the pharmacological agents capable capable of down regulating REST (the molecular barrier to trans-differentiation) therefore capable of enhancing differentionation.

We found supplementation of fibroblast neuronal transdifferentiation media with docosahexaenoic and eicosapentaenoic acids (DHA/EPA-IM) dramatically increased transdifferentiation efficiency from 20 to near 90% and accelerated transdifferentiation of non-neuronal cells, such as fibroblast and stem cells, into neurons from three weeks to less than a week.

Without wishing to be bound by theory, complexes of transcription factors, such as RE1-silencing transcription factor (REST), is a target for the media as described herein, a molecular mechanism by which transdifferentiation efficiency is increased. For example, DHA/EPA reduces expression and/or protein levels of transcription factors, such as REST. Such transcription factors are responsible for the repression of the expression of neuronal-specific genes in non-neuronal cells (Conaco C, Otto S, Han J J, Mandel G. Reciprocal actions of REST and a microRNA promote neuronal identity. Proc Natl Acad Sci U S A. 2006 Feb 14;103(7):2422-7. PMID: 16461918). By candidate gene approach we identified REST or Repressor element-1 (RE-1)-silencing transcription factor, also named neuron-restrictive silencer factor (NRSF) as the molecular barrier to transdifferentiation

Xue Y, Ouyang K, Huang J, Zhou Y, Ouyang H, Li H, Wang G, Wu Q, Wei C, Bi Y, Jiang L, Cai Z, Sun H, Zhang K, Zhang Y, Chen J, Fu X D. Direct conversion of fibroblasts to neurons by reprogramming PTB-regulated microRNA circuits. Cell. 2013; 152: 82-96. PMID:23313552

Masserdotti G, Gillotin S, Sutor B, Drechsel D, Irmler M, Jorgensen H F, Sass S, Theis F J, Beckers J, Berninger B, Guillemot F, Gotz M. Transcriptional Mechanisms of Proneural Factors and REST in Regulating Neuronal Reprogramming of Astrocytes. Cell Stem Cell. 2015; 17: 74-88. PMID: 26119235

[symbol]

Neuronal induction medium:

DMEM/F12, Life Technologies, 11330-032: (Neurobasal, Life Technologies, 21103-049) (1:1), 0.5% N-2 (lnvitrogen, 17502048), 1% B-27 (Invitrogen, 17504044), 100 μM cAMP and 20 ng/ml bFGF and penicillin/streptomycin. VPA, 0.5 mM; CHIR99021, 3 μM; Repsox, 1 μM; Forskolin, 10 μM; SP600125, 10 μM; G06983, 5 μM; Y-27632, 5 μM.

[symbol]

DHA/EPA Induction Media (DHA/EPA-IM):

DMEM/F12, Life Technologies, 11330-032):(Neurobasal, Lifer Technologies, 21103-049 (1:1), 0.5% N-2 (Invitrogen, 17502048), 1% B-27 (Invitrogen, 17504044), 100 μM cAMP and 20 ng/m1 bFGF and penicillin/streptomycin. VPA, 0.5 mM; CHIR99021 3 μM; Repsox, 1 μM; Forskolin, 10 μM; SP600125, 10 μM; G06983, 5 μM; Y-27632, 5 μM μM; G06983, 5 μM; Y-27632, 5 μM, 20 μM EPA; 40 μM DHA.

### References Cited in This Example

1—Takahashi K, Yamanaka S. Induction of pluripotent stem cells from mouse embryonic and adult fibroblast cultures by defined factors. Cell. 2006; 126: 663-676. PMID: 16904174.

2—Vierbuchen T, Ostermeier A, Pang Z P, Kokubu Y, Sudhof T C, Wernig M. Direct conversion of fibroblasts to functional neurons by defined factors. Nature. 2010; 463: 1035-1041. PMID: 20107439.

3—Caiazzo M1, Dell'Anno M T, Dvoretskova E, Lazarevic D, Taverna S, Leo D, Sotnikova T D, Menegon A, Roncaglia P, Colciago G, Russo G, Carninci P, Pezzoli G, Gainetdinov R R, Gustincich S, Dityatev A, Broccoli V. Direct generation of functional dopaminergic neurons from mouse and human fibroblasts. Nature. 2011 Jul 3;476(7359):224-7. PMID: 21725324

4—Pfisterer U1, Kirkeby A, Torper O, Wood J, Nelander J, Dufour A, Bjorklund A, Lindvall O, Jakobsson J, Parmar M. Direct conversion of human fibroblasts to dopaminergic neurons. Proc Natl Acad Sci U S A. 2011 Jun 21;108(25):10343-8. PMID: 21646515.

5—Marro S1, Pang Z P, Yang N, Tsai M C, Qu K, Chang H Y, Südhof T C, Wernig M. Direct lineage conversion of terminally differentiated hepatocytes to functional neurons. Cell Stem Cell. 2011 Oct 4;9(4):374-82. PMID: 21962918.

6—Wapinski OL1, Vierbuchen T, Qu K, Lee Q Y, Chanda S, Fuentes D R, Giresi P G, Ng Y H, Marro S, Neff N F, Drechsel D, Martynoga B, Castro D S, Webb A E, Südhof T C, Brunet A, Guillemot F, Chang H Y, Wernig M. Hierarchical mechanisms for direct reprogramming of fibroblasts to neurons. Cell. 2013 Oct 24;155(3):621-35. PMID: 24243019.

7—Wang YJ1, Herlyn M2. The emerging roles of Oct4 in tumor-initiating cells. Am J Physiol Cell Physiol. 2015 Dec 1;309(11):C709-18. PMID: 26447206.

8—Pandya A Y, Talley L I, Frost A R, Fitzgerald T J, Trivedi V, Chakravarthy M, Chhieng D C, Grizzle W E, Engler J A, Krontiras H, Bland Kl, LoBuglio A F, Lobo-Ruppert S M, Ruppert J M. Nuclear localization of KLF4 is associated with an aggressive phenotype in early-stage breast cancer. Clin Cancer Res. 2004; 10: 2709-27019. PMID: 15102675.

9—Bilir B, Osunkoya A O, Wiles W G 4th, Sannigrahi S, Lefebvre V, Metzger D, Spyropoulos D D, Martin W O, Moreno C S. SOX4 is Essential for Prostate Tumorigenesis Initiated by PTEN Ablation. Cancer Res. 2016; 76: 1112-11121. PMID: 26701805.

10—Lastowska M, Al-Afghani H, Al-Balool H H, Sheth H, Mercer E, Coxhead J M, Redfern C P, Peters H, Burt A D, Santibanez-Koref M, Bacon C M, Chesler L, Rust A G, Adams D J, Williamson D, Clifford S C, Jackson M S. Identification of a neuronal transcription factor network involved in medulloblastoma development. Acta Neuropathol Commun. 2013; 1:35. PMID: 24252690.

11—Meder L, Konig K, Ozretic L, Schultheis A M, Ueckeroth F, Ade C P, Albus K, Boehm D, Rommerscheidt-Fuss U, Florin A, Buhl T, Hartmann W, Wolf J, Merkelbach-Bruse S, Eilers M, Perner S, Heukamp L C, Buettner R. NOTCH, ASCL1, p53 and RB alterations define an alternative pathway driving neuroendocrine and small cell lung carcinomas. Int J Cancer. 2016; 138: 927-938. PMID: 26340530.

12—Thomson, J. A. F., Murphy, K., Baker, E., Sutherland, G. R., Parsons, P. G., and Sturm, R A. The brn-2 gene regulates the melanocytic phenotype and tumorigenic potential of human melanoma cells. Oncogene. 1995; 11: 691-700.

13—Cook A L, Sturm R A. POU domain transcription factors: BRN2 as a regulator of melanocytic growth and tumourigenesis. Pigment Cell Melanoma Res. 2008; 21: 611-626. PMID: 18983536.

14—Hou P1, Li Y, Zhang X, Liu C, Guan J, Li H, Zhao T, Ye J, Yang W, Liu K, Ge J, Xu J, Zhang Q, Zhao Y, Deng H. Pluripotent stem cells induced from mouse somatic cells by small-molecule compounds. Science. 2013 Aug 9;341(6146):651-4. PMID: 23868920.

15—Hu W, Qiu B, Guan W, Wang Q, Wang M, Li W, Gao L, Shen L, Huang Y, Xie G, Zhao H, Jin Y, Tang B, Yu Y, Zhao J, Pei G. Direct conversion of normal and Alzheimer's disease human fibroblasts into neuronal cells by small molecules. Cell Stem Cell. 2015; 17:204-212. PMID: 26253202.

16—Eady TN1, Belayev L, Khoutorova L, Atkins K D, Zhang C, Bazan N G. Docosahexaenoic acid signaling modulates cell survival in experimental ischemic stroke penumbra and initiates long-term repair in young and aged rats. PLoS One. 2012;7(10):e46151. PMID:23118851.˜

17—Dyall SC1, Michael G J, Michael-Titus A T. Omega-3 fatty acids reverse age-related decreases in nuclear receptors and increase neurogenesis in old rats. J Neurosci Res. 2010 Aug 1;88(10):2091-102. PMID: 20336774.

18—Bazan NG1, Molina M F, Gordon W C. Docosahexaenoic acid signalolipidomics in nutrition: significance in aging, neuroinflammation, macular degeneration, Alzheimer's, and other neurodegenerative diseases. Annu Rev Nutr. 2011 Aug 21;31:321-51. PMID: 21756134

19—Mukherjee PK1, Marcheselli V L, Serhan C N, Bazan N G. Neuroprotectin D1: a docosahexaenoic acid-derived docosatriene protects human retinal pigment epithelial cells from oxidative stress. Proc Natl Acad Sci U S A. 2004 Jun 1;101(22):8491-6. PMID: 15152078

20—Hong S1, Tian H2, Lu Y2, Laborde JM3, Muhale FA2, Wang Q2, Alapure BV2, Serhan CN4, Bazan NG5Neuroprotectin/protectin D1: endogenous biosynthesis and actions on diabetic macrophages in promoting wound healing and innervation impaired by diabetes. Am J Physiol Cell Physiol. 2014 Dec 1;307(11):C1058-67. PMID: 25273880.

21—Calandria JM1, Asatryan A1, Balaszczuk V1, Knott EJ1 Jun BK1, Mukherjee PK1, Belayev L1, Bazan NG1. NPD1-mediated stereoselective regulation of BIRC3 expression through cREL is decisive for neural cell survival. Cell Death Differ. 2015 Aug;22(8):1363-77. PMID: 25633199.

22—Katakura M, Hashimoto M, Okui T, Shandat H M, Matsuzaki K, Shido 0. Omega-3 polyunsaturated Fatty acids enhance neuronal differentiation in cultured rat neural stem cells. Stem Cells Int. 2013; 490476. PMID: 23365582.

### Example 2

Chemical-based cell culture for direct conversion from other cells: Stem cells prepared in this media rescue cortical or hippocampal neurons after glucose plus oxygen deprivation

Composition of Trans-differentiation Media:

The base trans-differentiation media (Induction Medium, IM) was published by Hu et al., 2015 (PMID: 26253202). It is a chemical cocktail composed of V, Valproic Acid 0.5 mM (#P4543, SIGMA); C, CHIR99021, 3 μM, (#4423, Tocris, Minneapolis, Minn.); R, Repsox, 1 μM (#3742, Tocris); F, Forskolin, 10 μM, (#1099, Tocris); S, SP600125, 10 μM (#1496, Tocris); G, GO6983, 5 μM (#2285, Tocris); Y, Y-27632, 5 μM, (#1254, Tocris) added to adherent cells dispersed in (DMEM/F12, Life Technologies, #11330-032): (Neurobasal, Life Technologies, #21103-049) (1:1), 0.5% N-2 (Invitrogen, 17502048), 1% B-27 (Invitrogen, 17504044), 100 μM cAMP and 20 ng/ml of fibroblast growth factor-basic (bFGF, #03-0002, STEMGENT, LEXINGTON, Mass.) and Penicillin, Streptomycyn and Antimycotic (Gibco).

As described herein, a modified induction medium (also referered to as BM) comprises supplementing the basic IM with two essential fatty acids, namely 10 μM of eicosapentaenoic acid, (EPA, 20:5 n-3) and 20 μM docosahexaenoic acid (DHA, 22:6 n-3).

Preparation of rat primary neurons

All surgical procedures to isolated cortical and hippocampal primary neurons were performed under sterile conditions in a Nuaire Laminar Flow hood (Nuaire, Plymouth, Minn.). Timed pregnant embryonic day 18 (E18) Sprague Dawley rats (Charles River Laboratories) were euthanized and embryos harvested, sacrificed and brains aseptically collected and placed in petri dish containing ice-cold Hanks' Balanced Salt Solution, 1× (HBSS, #21-020-CV, Mediatech). Cortices and hippocampus were surgically isolated under a Nikon SMZ 645 stereomicroscope (Nikon, Melville, N.Y.). Tissues were chopped into small pieces with the help of micro-spring scissors, transferred to 15 ml Corning CentriStar centrifuge tubes (#430791, Corning, Corning, N.Y.). Next, HBSS containing 0.25% v/v trypsin (#15090-046, Gibco) and 0.25% w/v deoxyribonuclease I from bovine pancreas (DNase I, #DN25, SIGMA, St. Louis, Mo.) was added, then tubes placed in a water bath with agitation. After 15 minutes, FBS was added to 10%v/v (final concentration) to stop digestion. These tissues were subsequently triturated 15 times with a fire polished Pasteur pipet and left 2 minutes to allow debris to settle down. Supernatant was transferred to a new 15 ml centrifuge tube (Corning), then filtered through a 70 μm pore-sized Corning cell Strainer (#431751, Corning) and cells pelleted in a refrigerated Eppendorff 5840 R centrifuge (Eppendorff, Hauppauge, N.Y., 5 minutes, 1000 rpm at 4° C.). Cell pellets were resuspended in Neurobasal medium (GIBCO) supplemented with 2% v/v each B-27 and N2, along with 1× each Glutamax (#35050-061, Gibco) and penicillin/streptomycin (#15070063, Gibco). Cells were counted using a Neubauer hemocytometer under 10× objective utilizing a Nikon Eclipse TS100 microscope (Nikon). Cortical and hippocampal neurons were seeded at the densities of ˜2.5×105 and ˜1.5×105 cells/well respectively on TPP® tissue culture 12-well plates pre-coated with 25 μg/ml poly-L-lysine hydrobromide as described above and incubated at 37° C., 5% CO2 humid atmosphere. The following media was aspirated and replaced with fresh pre-warmed complete neurobasal. Subsequently, every three days half of the media was replaced with fresh medium.

Neuronal Immunophenotyping

Neuronal and stem cells cultures were characterized by immunofluorescence staining with the class-III-β tubulin monoclonal antibody, the rabbit anti-glial fibrillary acidic protein, the doublecortin (#T8660 and #G9269, # D9818 all from SIGMA). In addition we used the Millipore monoclonal antibodies anti-NeuN (#MAN377) and anti-Map2 (#MAB3418) and the the rabbit monoclonal antibodies anti-synapsin (#5297) and synaptophysin (# 5461) all from CellSignal. Bound primary antibodies were detected following incubation with AF488 conjugated donkey anti-mouse IgG (#A-21202, Thermofisher) and AF647 conjugated donkey anti-rabbit (#A-31573) and nuclei counterstained with Hoechst 33258 (#94403, SIGMA). Briefly, cells were fixed with 4% paraformaldehyde, permeabilizead with 0.1% v/v Triton ×100, and inhibition of non-specific binding done with 5% bovine serum albumin in PBS containing 0.05%v/v Tween-20 PBS-T). Following overnight incubation, bound primary antibodies were detected with species specific secondary antibody conjugated with fluorophore as indicated above. Nuclear staining was done with 0.2 μM Hoescht. Were applicable washes were performed three times with PBS-T.

Oxygen Glucose Deprivation (OGD)

To mimic stroke in vitro, two weeks-old rat neuronal primary cultures monolayers were washed with pre-warmed phosphate-buffered saline and pre-incubated in the presence of glucose-, sodium pyruvate-, and glutamine free Neurobasal medium (#A24775-01, Gibco) during 30 minutes. Thereafter, plates were placed in a modular incubator chamber (#MIC-101, Billups-Rothenberg, Del Mar, Calif.) and air was replaced with an anaerobic mixture of gases (95% N2, 5% CO2, #1956, Air Gas, Radnor, Pa.) before placing in the incubator at 37° C. and anaerobic humid atmosphere.

In vitro Cell-based Rescue of Rat Primary Neurons from Programmed Cell Death After Insult by Nutrient and Oxygen Deprivation

One hour after OGD, cells were returned to complete neurobasal medium alone, or with 1:1, NB complete: conditioned media before being placed in normoxic humid atmosphere with 5% CO2.

Assessment of Cell Viability by AlamarBlue

Three days after OGD, alamarBlue® Cell Viability Reagent (#DAL1100, Life Technologies) was added (10% v/v). Rates of reduction of non-fluorescent blue-colored resazurin into highly fluorescent red-colored resorfurin were measured in a Spectramax M5 plate reader (excitation 530 nm/emission 590 nm, Molecular Devices, Sunnyvale, Calif.). For fluorescence readings of, two hundred microliters of media samples were collected and transferred from the experimental wells into Corning™ 96-Well Clear Bottom Black Polystyrene Microplate (#3603, Corning).

Statistical Analysis

Experimental values are expressed as mean ±SD from triplicates and significance was set at p value ≤0.05 with the one sided Student's t-test.

Results

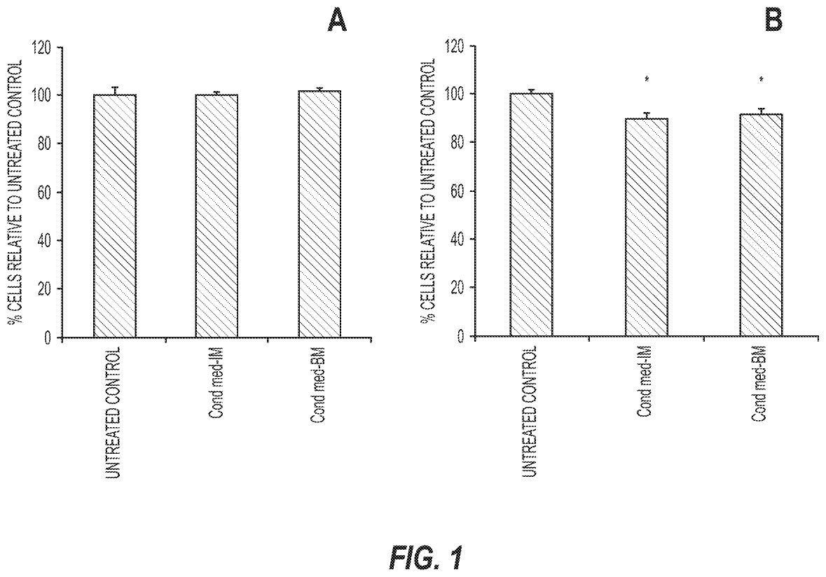

Z′ factor for the alamarBlue assay was ≥0.9 which shows that it a very strong and robust assay. In the absence of injury there were no differences in cell numbers irrespectively to the type of growth media for the cortical neurons (FIG. 1A). However hippocampal neuron cultures had a slight but statistically significant decrease on cell number relative to controls grown in complete NB media (FIG. 1B).

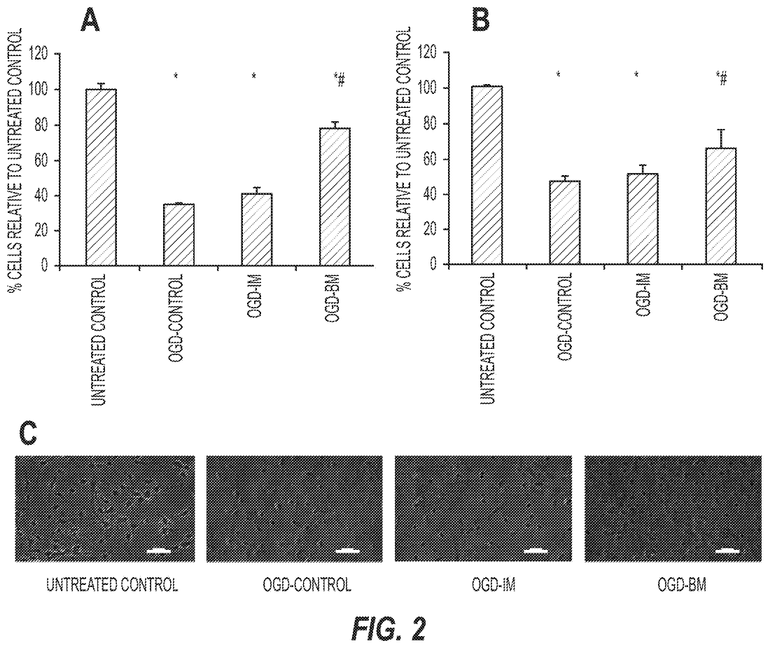

Three days after OGD, cell populations had decrease by about 60% in both cortical and hippocampal neurons grown in either the regular complete NB or IM media (FIG. 2). These observations indicate that our in vitro system to mimic stroke was effective and that neither of these two media possess cytoprotective proprieties. Our new BM media showed a robust and statistically significant neuroprotection for both types of neuronal primary cultures (FIG. 2). Neurons transdifferentiated with BM (but not IM) excrete products in the conditioned media that are cytoprotective. This result shows a paracrine effect of the transdifferentiated neurons on injured rat neurons. Since regular neuro-basal and IM performed similarly we can consider that the chemicals we used to promote trans-differentiation of fibroblasts into neurons have little or no ability to rescue neurons from uncompensated oxidative stress following OGD. Conversely the addition of DHA and EPA (new BM media) may have provided transdifferentiated neurons with substrates to generate and excrete into the media mediators and/or exosomes that exert protection.

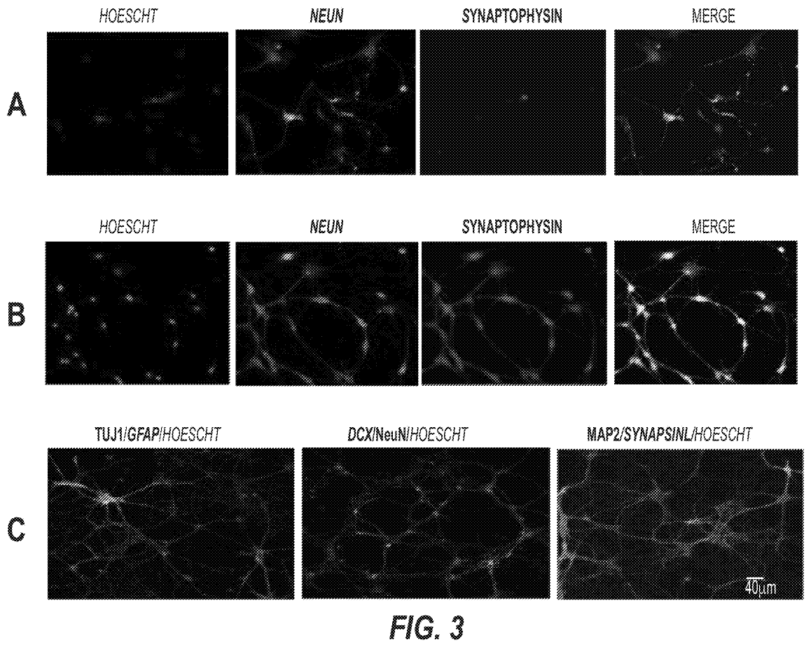

FIG. 3 shows immunofluorescence staining for two neuron specific markers NeuN and synaptophysin. Briefly, cells were fixed with 4% w/v paraformaldehyde, permeabilizead with 0.1% v/v Triton ×100, and inhibition of non-specific binding done with 5% bovine serum albumin in PBS containing 0.05%v/v Tween-20 PBS-T). Following overninght incubation, bound primary antibodies were detected with species specific secondary antibody conjugated with fluorophore as indicated above. Nuclear staining was done with 0.2 μM Hoescht. Were applicable washes were performed three times with PBS-T.

[symbol]

In the BM-induced group the relative number of immunoreactive cells and intensity of staining is much greater compared to IM-induced. Therefore, BM is more effective inducer of chemical neuronal trans-differentiation. In addition, our data also show that rat neurons used for the rescue experiments possessed the cognate morphological and immunostaining patterns.

### Example 3

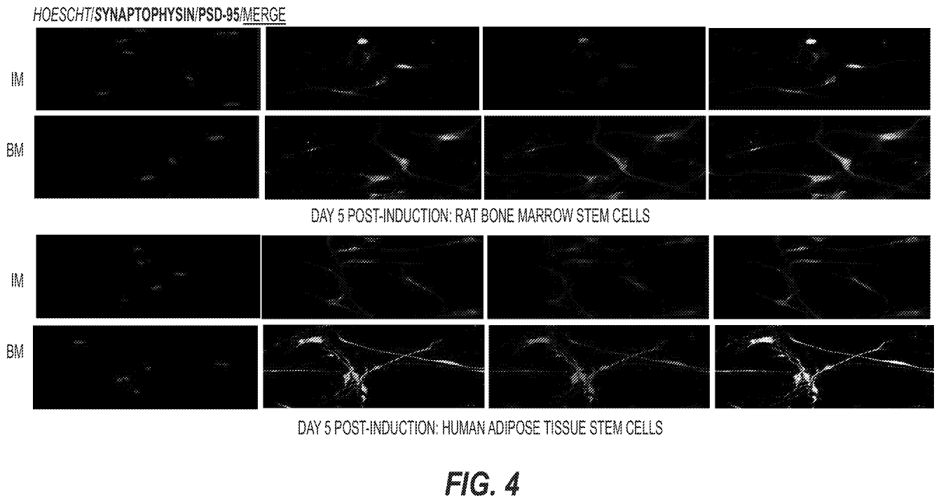

We successfully extended the use of neuronal-type chemical induction to both types of mesenchymal stem cells either derived from rat bone marrow or from the human adipose tissue (FIG. 4). Mesenchymal stem cells were cultured in DMEM with 10% FBS from the aspirates from tibias and femurs of adult rats (Soleimani M, Nadri S. A protocol for isolation and culture of mesenchymal stem cells from mouse bone marrow. Nat Protoc. 2009;4(1):102-6. PMID: 19131962. Human adipose tissue derived stem cells were purchased from Zen-Bio (RTP, NC).

Exponentially growing skin-derived human or mouse fibroblasts, mesenchymal stem cells derived from adipose tissue (human) or bone marrow (mouse) are seeded in 24-well plates at the density of ˜2.5×10⁵ cells per well. Growing media for fibroblasts is RPMI 1640 and DMEM is the media for the mesenchymal stem cells. All growing media are supplemented with 10% FBS, 1× each NEAA, sodium pyruvate, glutamine and antibiotics/antimycotic. Cultures are maintained in 37° C. incubator with 5% CO₂ humid atmosphere. Twenty four to forty eight hours post platting, growing media is replaced with pre-warmed freshly prepared IM or BM and plates put back into the incubator. Morphological changes are followed daily with the help of bright-field microscopy. By five days post-induction of transdifferentiation, clear morphological features of neuronal-type transdifferentiation become evident in both types of stem cells (FIG. 4). However transdifferentiation with induction media alone (IM) results into delayed maturation compared to induction media containing 10 μM EPA+20 μM DHA (BM).

We used immunofluorescence labeling followed by confocal microscopy analysis for two functional neuronal markers synaptophysin and post synaptic density 95 (PSD-95). We found that BM-treated stem cells present more complex dendritic network and all cells showed strong positive immunostaining. However not all cells induced with IM expressed these two proteins and when present the staining is sparse and weaker.

### Example 4

Flow of the ω-3 fatty acid (FA), docosahexaenoic acid (DHA) (22:6), or its precursor, α-linolenic acid (ALA) (18:3), from the gastrointestinal lumen to the lymphatic system.

DHA is required for the synthesis of phospholipids for nervous system membrane biogenesis. If unavailable, DHA can be synthesized from ALA. Enterocytes of the small intestine take up these FAs and package them for delivery to the endothelial cells of the lymphatic system. From there, they are transferred to the central lacteal and delivered to the circulatory system for transport. Detailed molecular events are not well understood as of yet.

Desaturation and elongation of α-linolenic acid (ALA) (18:3) within the hepatocyte.

Systemic ALA is taken up by hepatocytes and transferred to the endoplasmic reticulum, where a series of desaturation and elongation steps occurs, leading to the formation of a 24-carbon, 6-double-bond FA [24:6, tetracosahexaenoic acid (THA)]. 24:6 is then conveyed to peroxisomes, converted to 22:6 by β-degradation, and delivered back to the endoplasmic reticulum. 22:6 (DHA) is then attached to the n-2 position of phosphatidyl choline to form a 22:6 phospholipid (22:6-PL), followed by release to the circulatory system for delivery to the choriocapillaris behind the retina and neovascular unit within the brain, as well as to other tissues.

Movement of docosahexaenoic acid (DHA) (22:6) through the neurovascular unit and its disposition within neurons and astrocytes.

22:6-phospholipids (22:6-PLs) are taken up by endothelial cells from the circulation and transferred to astrocytes within the central nervous system; from there, 22:6 is incorporated into astrocytes or transferred to neurons. 22:6 is also available for conversion to neuroprotectin D1 (NPD1) upon inductive signals. After packaging in the endoplasmic reticulum and the Golgi apparatus, 22:6 is also transported to the synaptic terminal to become incorporated into synaptic elements. Arrows represent possible routes. Molecular characterization of transporter(s) and receptors remains to be done.

Docosahexaenoic acid (DHA) (22:6) delivery to photoreceptors, photoreceptor membrane renewal, and synthesis of neuroprotectin D1 (NPD1).

DHA (22:6) or immediate precursors are obtained by diet. Once within the liver, hepatocytes incorporate 22:6 into 22:6-phospholipid (22:6-PL)-lipoproteins, which are then transported to the choriocapillaris. 22:6 crosses Bruch's membrane from the subretinal circulation and is taken up by the retinal pigment epithelium (RPE) cells lining the back of the retina to subsequently be sent to the underlying photoreceptors. This targeted delivery route from the liver to the retina is referred to as the 22:6 long loop. Then 22:6 passes through the interphotoreceptor matrix (IPM) and into the photoreceptor inner segment, where it is incorporated into phospholipids for cell membrane, organelles, and disk membrane biogenesis. As new 22:6-rich disks are synthesized at the base of the photoreceptor outer segment, older disks are pushed apically toward the RPE cells. Photoreceptor tips are phagocytized by the RPE cells each day, removing the oldest disks. The resulting phagosomes are degraded within the RPE cells, and 22:6 is recycled back to photoreceptor inner segments for new disk membrane biogenesis. This local recycling is referred to as the 22:6 short loop. Upon inductive signaling, such as pigment-epithelium derived factor (PEDF), 22:6 can be obtained from a phospholipid pool for the synthesis of neuroprotectin D1 (NPD1).

Biosynthesis of neuroprotectin D1 (NPD1).

A membrane phospholipid containing a docosahexaenoyl chain at sn-2 is hydrolyzed by phospholipase A2, generating free (unesterified) docosahexaenoic acid (DHA) (22:6). Lipoxygenation is then followed by epoxidation and hydrolysis, to generate NPD1. Thus far, a binding site for NPD1 has been identified in retinal pigment epithelium cells and polymorphonuclear cells (133). BDNF, brain-derived neurotrophic factor; CNTF, ciliary neurotrophic factor; CEX-1, cytokine exodus protein-1; COX-2, cyclooxygenase-2; LIF, leukemia inhibitory factor; NT3, neurotrophin 3; PEDF, pigment epithelium-derived factor.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A cell culture medium composition, the composition comprising an effective amount of: valproic acid; a GSK-3 inhibitor; a TGFβR1 inhibitor; Forskolin; a JNK inhibitor; a protein kinase C (PKC) inhibitor; a Rho-associated, coiled-coil containing protein kinase (ROCK) inhibitor; at least one omega-3 fatty acid; and a nutrient source, wherein
the amount of Valproic Acid ranges from about 0.5 mM to about 1 mM;
the amount of a GSK-3 inhibitor ranges from about 1 μM to about 10 μM;
the amount of a TGFβR1 inhibitor ranges from about 1 μM to about 10 μM;
the amount of Forskolin ranges from about 1 μM to about 10 μM;
the amount of a JNK inhibitor ranges from about 1 μM to about 10 μM;
the amount of a PKC inhibitor ranges from about 1 μM to about 10 μM;
the amount of a ROCK inhibitor ranges from about 1 μM to about 10 μM;
and the amount of an omega-3 fatty acid ranges from about 1 μM to about 20 μM.

2. The composition of claim 1, wherein the amount of Valproic Acid comprises about 0.5 mM.

3. The composition of claim 1, wherein the GSK-3 inhibitor comprises CHIR99021.

4. The composition of claim 1, wherein the amount of GSK-3 inhibitor comprises about 3 μM.

5. The composition of claim 1, wherein the TGFβR1 inhibitor comprises Repsox, A8-301, or an ALK-5 inhibitor.

6. The composition of claim 1, wherein the amount of TGFβR1 inhibitor comprises about 1 μM.

7. The composition of claim 5, wherein the ALK-5 inhibitor comprises SB431542.

8. The composition of claim 1, wherein the amount of Forskolin comprises about 10 μM.

9. The composition of claim 1, wherein the JNK inhibitor comprises SP600125.

10. The composition of claim 1, wherein the amount of JNK inhibitor comprises about 10 μM.

11. The composition of claim 1, wherein the PKC inhibitor comprises GO6983 or H7.

12. The composition of claim 1, wherein the amount of PKC inhibitor comprises about 5 μM.

13. The composition of claim 1, wherein the ROCK inhibitor comprises Y-27632, 1-(5-Isoquinolinesulfonyl) homopiperazine, N-Benzyl-2-(pyrimidin-4-ylamino) thiazole-4-carboxamide, (+)-(R)-trans-4-(1-aminoethyl)-N-(4-pyridyl) cyclo-hexanecarboxamide dihydrochloride) and N-{(3R,4R)-4-[4-(2-Fluoro-6-hydroxy -3-methoxy-benzoyl)-benzoylamino]-azep-an-3-yl}-4-hydroxy-3,5-dimethyl-benzamide.

14. The composition of claim 1, wherein the amount of ROCK inhibitor comprises about 5 μM.

15. The composition of claim 1, wherein the omega-3 fatty acid is selected from at least one of Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA), Clupanodonic acid, Docosahexaenoic acid (DHA), Tetracosapentaenoic acid, Tetracosahexaenoic acid (Nisinic acid), or precursors thereof.

16. The composition of claim 1, wherein the omega-3 fatty acid comprises eicosapentaenoic acid (EPA), docosahexaenoic acid (DHA), or a combination thereof.

17. The composition of claim 16, wherein the effective amount of EPA ranges from 1 μM to about 20 μM.

18. The composition of claim 16, wherein the effective amount of EPA is about 10 μM.

19. The composition of claim 16, wherein the effective amount of DHA ranges from about 1 μM to about 20 μM.

20. The composition of claim 16, wherein the effective amount of DHA is about 20 μM.

21. The composition of claim 1, wherein the nutrient source is selected from at least one of DMEM, IDMEM, MEM, M199, RPMI 1640, Ham's F12, DMEM/F-12, Ham's F10, McCoy's 5A, NCTC 109, and NCTC 135.

22. The composition of claim 1, wherein the nutrient source comprises DMEM.

23. A method of promoting neuronal cell conversion, the method comprising:
obtaining a plurality of non-neuronal cells;
admixing the plurality of non-neuronal cells with the cell culture medium composition of any one of claims 1-22; and
culturing the admixture for a period of time sufficient for the non-neuronal cells to transdifferentiate into neuronal cells.

24. A method of inducing neuronal cell differentiation, the method comprising:
obtaining a plurality of non-neuronal cells;
admixing the plurality of non-neuronal cells with the cell culture medium composition of any one of claims 1-22; and
culturing the admixture for a period of time sufficient to induce differentiation of non-neuronal cells into neuronal cells.

25. A method of treating a subject afflicted with a disease characterized by neuronal death, neuronal injury, or both, the method comprising:
obtaining a plurality of non-neuronal cells;
admixing the plurality of non-neuronal cells with the cell culture medium composition of any ones of claim 1-22;
culturing the admixture for a period of time sufficient to induce differentiation of non-neuronal cells into neuronal cells; and
administering the differentiated neuronal cells to the subject in need thereof.

26. The method of claims 23, 24, and 25, wherein differentiation into neuronal cells is detected by immunophenotyping.

27. The method of claim 25, wherein the plurality of non-neuronal cells have been isolated from the subject.

28. The method of any of claim 23, 24, or 25, wherein the neuronal cells comprise functional neurons.

29. The method of any one of claim 23, 24, or 25, wherein the period of time comprises about 1, 2, 3, 4, 5, 6, 7, 8, 9, or 10 days.

30. The method of any one of claim 23, 24, or 25, wherein the period of time is less than 21 days.

31. The method of any one of claim 23, 24, or 25, wherein the non-neuronal cells comprise fibroblasts or mesenchymal stems cells

32. The method of claim 24 or 25, further comprising administering the neuronal cells to a subject afflicted with a disease characterized by neuronal death, neuronal injury, or both.

33. The method of claim 32, wherein the disease comprises a neurological disorder.

34. The method of claim 32, wherein the neurological disorder comprises stroke, Alzheimer's Disease, Parkinson's Disease, Amyotrophic Lateral Sclerosis (ALS), Multiple Sclerosis (MS), Frontotemporal dementia, and Huntington's disease.

35. The method of claim 32, wherein the neurological disorder comprises a neurodegenerative disorder.

36. An in vitro culture comprising an isolated population of in vitro transdifferentiated cells and a serum-free cell culture medium supplemented with DHA and/or EPA, wherein greater than 90% of the cells in the population are neuronal cells.

37. A cytoprotective conditioned media composition produced by transdifferentiated neurons cultured in the media of claim 1 for a period of time.

38. The composition of claim 37, wherein the media is a serum-free cell culture medium.

39. The composition of claim 37, further comprising docosanoids, NPD1, maresins, resolvins, or a combination thereof

40. A method of treating a subject afflicted with a disease characterized by neuronal death, neuronal injury, or both, the method comprising administering a therapeutically effective amount of the cytoprotective conditioned media of claim 37 to the subject in need thereof.

41. A method of delaying or preventing neuronal degeneration in a subject suffering from a neurological disorder, the method comprising administering to the subject a therapeutically effective amount of the conditioned media of claim 37 to the subject.

42. A method of treating a subject afflicted with a neurological disorder, the method comprising administering a therapeutically effective amount of the conditioned media of claim 37 to the subject.

43. The method of claim 41 or 42, wherein the neurological disorder comprises stroke, Alzheimer's Disease, Parkinson's Disease, Amyotrophic Lateral Sclerosis (ALS), Multiple Sclerosis (MS), Frontotemporal dementia, and Huntington's disease.

44. The method of claim 41 or 42, wherein the neurological disorder comprises a neurodegenerative disorder.

45. A method of making a cytoprotective conditioned media, the method comprising culturing transdifferentiated neurons in the media of claim 1 for a period of time sufficient for the media to develop cytoprotective properties.

46. The method of claim 45, wherein the cytoprotective properties of the media is indicated by the presence of at least one docosanoid, at least one maresin, at least one resolvin, or NPD1.
